(19) United States
(12) Patent Application Publication
(10) Pub. No.: US 2012/0116321 A1
(43) Pub. Date: May 10, 2012

(54) NEEDLE SET WITH SEPARABLE LINE TO REDUCE SHARPS WASTE

(75) Inventor: James M. Brugger, Newburyport, MA (US)

(73) Assignee: NxStage Medical, Inc., Lawrence, MA (US)

(21) Appl. No.: 13/322,566

(22) PCT Filed: May 27, 2010
(86) PCT No.: PCT/US10/36427

§ 371 Date: Jan. 25, 2012

Related U.S. Application Data
(60) Provisional application No. 61/181,373, filed on May 27, 2009.

(51) Int. Cl. A61M 5/00

(52) U.S. Cl. 604/263; 604/272

(57) ABSTRACT

A sharp medical device is attached to a flexible element by a base of the sharp device. The base has a portion with a score line defining a position of predictable failure such that the base can be broken by applying a breaking moment at the score line to permit a user to separate the sharp device and the flexible element cleanly at the position of predictable failure. Various other embodiments are included, which disclose a similar effect.

### CROSS-REFERENCE TO RELATED APPLICATIONS

This International Application claims the benefit of U.S. Provisional Application No. 61/181,373, filed on May 27, 2009.

### BACKGROUND

Sharps waste is a type of medical waste that can be hazardous and preferably is subjected to special handling procedures and system. The handling procedures and systems can involve unwelcome additional costs compared to the handling of non-sharp waste. There is a perennial need for reducing the volume of sharps waste.

### SUMMARY

Unitary molded structures can be broken apart to separate sharp medical devices from non-sharp parts without incurring additional manufacturing costs by forming frangible portions. A particular application is a needle set with preattached tubing.

According to embodiments, the disclosed subject matter includes a sharp medical device with a needle set having a needle portion including a cannula and a base. The base has wings and the cannula has an internal lumen. A tubing portion is attached to the needle portion base to form a continuous flow channel with the cannula internal lumen. A frangible connector is located inline with the tubing portion and configured to be broken cleanly. The frangible connector is integrally formed with the needle base portion. The frangible connector has extension along an axis of the tubular portion has a length of at least 1.5 cm. The frangible connector has a breaking moment that is sufficiently high that the needle base portion can be reliably gripped during use without breaking the frangible connector. A needle guard is slidably engaged with the tubing portion. The frangible connector has edges that are sloped to form a gradual diameter transition to prevent the needle guard from catching on edges of the frangible connector. The cannula and tube are sealed by respective sealing caps and they are sterile within a sealed interior. The tube portion a length of at least 20 cm is permanently attached from end to end without any intervening connectors.

According to embodiments, the disclosed subject matter includes a sharp medical device with a needle set having a needle portion including a cannula and a base, the cannula having an internal lumen. A tubing portion is attached to the needle portion base and forms a continuous flow channel with the cannula internal lumen. A frangible connector is located inline with the tubing portion and is configured to be broken cleanly. The cannula and tube are sealed by respective sealing caps and being sterile within a sealed lumen thereof. The frangible connector may be integral with the base. The entirety of the needle set from tubing portion through the cannula may be permanently connected with no intervening connectors. The cannula and tube may be sealed by respective sealing caps and a sealed interior thereof may be sterile. The cannula and tube may be sealed by respective sealing caps and being sterile within a sealed interior. The frangible connector may be integral with the base. The cannula may be a pheresis or fistula needle.

According to embodiments, the disclosed subject matter includes a method of using and disposing of a needle. The method includes providing a cannula and tubing portion with a common lumen therein where the tubing portion has an integral inline frangible connector. The method also includes sealing the ends of the lumen and sterilizing the cannula and tubing portion and delivering the cannula and tubing portion to a point of use. The method further includes inserting the cannula into an animal; removing the cannula from the animal. The method includes breaking the frangible connector; disposing of the cannula in a first waste container for medical sharps and disposing of the tubing portion in a second waste container for non-sharps medical waste. According to embodiments, the disclosed subject matter includes a sharp medical device with a sharp device attached to a non-sharp element. The sharp device is attached to the non-sharp element by a base of the sharp device. The base has a portion with a score line defining a position of predictable failure such that the base can be broken by applying a breaking moment at the score line to permit a user to separate the sharp device and the non-sharp element cleanly at the position of predictable failure. The sharp device may include a cannula and the non-sharp element may include a tube. The sharp device may be enclosed in a sterile container.

According to embodiments, the disclosed subject matter includes a method of using and disposing of a sharp medical device attached to a flexible portion, comprising: providing a sharp medical device with a non-sharp portion connected by a frangible connector; sterilizing the sharp medical device and enclosing in a sterile container; delivering the sterile container to a point of use and removing it from the sterile container; after use, breaking the frangible connector; disposing of the sharp medical device in a first waste container for medical sharps; and disposing of the non-sharp portion in a second waste container.

### BRIEF DESCRIPTION OF THE DRAWINGS

FIGS. 1A and 1B shows a first embodiment of a sharps waste reducing needle set in integral and separation conditions, respectively.

FIG. 2 shows another embodiment of a sharps waste reducing needle set.

### DETAILED DESCRIPTION OF EMBODIMENTS

Needles and other sharp medical waste must be deposited, after use, in a special “sharps container.” Such containers are provided in hospital and treatment centers for receiving sharp waste and involve elevated costs for handling and final disposal. The disposal of sharps involves significant costs.

Sharps often have an additional component beyond the sharp portion itself Such additional components can take up a large fraction of the space in a sharps container limiting the capacity of the sharps container and adding to the cost of disposal associated with the higher handling costs of sharp waste.

For example, fistula and pheresis needles and similar needles often have a pre-attached length of tubing. For sterility purposes, for convenience, or for other reasons, the preattachment of the tubing is desirable. Additional reasons that a preattached tube maybe provided are for use with a needle guard that slides over the tube when not covering a needle and to allow the needle guard to be slid over the needle when the needle is to be covered. Yet another reason for preattaching the tube to needles is to ensure against leaks or inadvertent disconnection of any intervening connector.

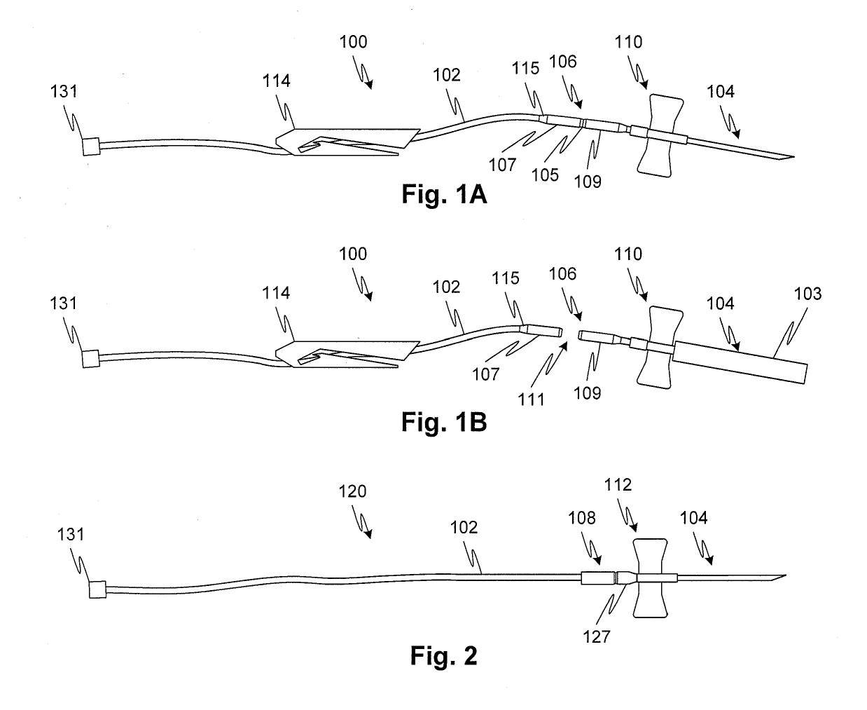

When fistula needles with preattached tubes are deposited in a sharps container, the attached tubing takes up a substantial volume of space in the sharps container. Referring to FIG. 1A, a fistula needle 100 has a tube length 102 preattached to a winged needle 110 with a sharp cannula 104. The tube length carries a frangible inline connector 106 which is made of a biocompatible material which, together with a shape thereof, causes it to break cleanly along a circumferential groove 105. The presence of the groove 105 and the sharp edges of the groove concentrate stress transmitted by a bending moment through tube portions 107 and 109. The bending moment may be supplied by a user by gripping the portions 107 and 109 (or respective portions of the embodiment of FIG. 2) which may be, for example, 1.5 cm long or longer to permit a bending moment to be applied thereto. Examples of suitable configurations and materials for making the connector frangible can be found in U.S. Pat. No. 5,279,605.

The inline connector 106 is preferably of a diameter and construction such that a needle guard 114 can be moved smoothly over the inline connector. For example, a smooth diameter transition portion 115 may be provided. Alternatively, the inline connector can be butt-welded to the tubing so that there is no change of outer diameter. Still other alternatives are possible, for example a lap-joint or fill-cement bond can be provided.

In the connector 106, the groove is circular which forms a break 111 as shown in FIG. 1B. Once broken, the tubing 102 portion can be disposed of in a non-sharps container and the needle 112 portion can be disposed of in a sharps container. This reduces the volume of waste required to be placed in the sharps container.

Referring to FIG. 2, a fistula or pheresis needle 120 with a winged needle 112 has a frangible connector 108 which forms part of the winged needle base 127. In an embodiment, the frangible connector 108 is integral to the winged needle.

The needle sets 100 and 120 may be sealed by a capped connector 131 and a needle cap 103 to seal a lumen therewithin. The entire needle set 100, 120 may then be sterilized as a unit. The needle cap 103 may be used with any of the embodiments described herein.

Instead of, or in addition to, providing gradual transitions on the inline connectors, the needle guard 114 can have sloped transitions of an internal passage that prevents the needle guard 114 from catching on the inline connector 106 or 108.

Although the embodiments described above, the needle sets are for fistula or pheresis needles, but other types of needle sets could be applications for the disclosed subject matter. For example, the sets could be used for infusion needle sets, medical treatment sets, dialysis needle sets, etc.

According to embodiments, the disclosed subject matter includes a sharp medical device, comprising: a needle set having a needle portion including a cannula and a base, the base having wings, the cannula having an internal lumen, a tubing portion attached to the needle portion base and forming a continuous flow channel with the cannula internal lumen, a frangible connector inline with the tubing portion and configured to be broken cleanly, the frangible connector being integrally formed with the needle base portion, the frangible connector having extension along an axis of the tubular portion having a length of at least 1.5 cm, the frangible connector having a breaking moment that is sufficiently high that the needle base portion can be reliably gripped during use without breaking the frangible connector, a needle guard slidably engaged with the tubing portion, the frangible connector having edges that are sloped to form a gradual diameter transition to prevent the needle guard from catching on edges of the frangible connector, the cannula and tube being sealed by respective sealing caps and being sterile within a sealed interior, the tube portion having a length of at least 20 cm and being permanently attached from end to end without any intervening connectors.

According to an embodiment, the disclosed subject matter includes a sharp medical device, comprising: a needle set having a needle portion including a cannula and a base, the cannula having an internal lumen, a tubing portion attached to the needle portion base and forming a continuous flow channel with the cannula internal lumen, a frangible connector inline with the tubing portion and configured to be broken cleanly, the cannula and tube being sealed by respective sealing caps and being sterile within a sealed lumen thereof.

In any of the embodiments, the frangible connector may be integral with the base. In any of the embodiments, the entirety of the needle set from tubing portion through the cannula may be permanently connected with no intervening connectors. In any of the embodiments, the cannula and tube may be sealed by respective sealing caps and a sealed interior thereof is sterile. In any of the embodiments, the cannula may be a pheresis or fistula needle or in other embodiments, the tubing set may be an infusion tubing set.

According to embodiments of the disclosed subject matter, a method of using and disposing of a needle, includes: providing a cannula and tubing portion with a common lumen therein, tubing portion having an integral inline frangible connector, sealing the ends of the lumen and sterilizing the cannula and tubing portion, delivering the cannula and tubing portion to a point of use, inserting the cannula into an animal, removing the cannula from the animal, breaking the frangible connector, disposing of the cannula in a first waste container for medical sharps, disposing of the tubing portion in a second waste container for non-sharps medical waste.

Having now described embodiments of the disclosed subject matter, it should be apparent to those skilled in the art that the foregoing is merely illustrative and not limiting, having been presented by way of example only. Numerous modifications and other embodiments (e.g., combinations, rearrangements, etc.) as well as features can be combined, rearranged, etc. within the scope of the invention to produce more embodiments.

1. A sharp medical device, comprising:
a needle set having a needle portion including a cannula and a base, the base having wings, the cannula having an internal lumen;
a tubing portion attached to the needle portion base and forming a continuous flow channel with the cannula internal lumen;
a frangible connector inline with the tubing portion and configured to be broken cleanly;
the frangible connector being integrally formed with the needle base portion, the frangible connector having extension along an axis of the tubular portion having a length of at least 1.5 cm;
the frangible connector having a breaking moment that is sufficiently high that the needle base portion can be reliably gripped during use without breaking the frangible connector;
a needle guard slidably engaged with the tubing portion;
the frangible connector having edges that are sloped to form a gradual diameter transition to prevent the needle guard from catching on edges of the frangible connector;
the cannula and tube being sealed by respective sealing caps and being sterile within a sealed interior;
the tube portion having a length of at least 20 cm and being permanently attached from end to end without any intervening connectors.

2-7. (canceled)

8. The device of claim 1, wherein the cannula is a pheresis or fistula needle.

9. A method of using and disposing of a needle, comprising:
providing a cannula and tubing portion with a common lumen therein, tubing portion having an integral inline frangible connector;
sealing the ends of the lumen and sterilizing the cannula and tubing portion;
delivering the cannula and tubing portion to a point of use;
inserting the cannula into an animal;
removing the cannula from the animal;
breaking the frangible connector;
disposing of the cannula in a first waste container for medical sharps;
disposing of the tubing portion in a second waste container for non-sharps medical waste.

10-12. (canceled)

13. A method of using and disposing of a sharp medical device attached to a flexible portion, comprising:
providing a sharp medical device with a non-sharp portion connected by a frangible connector;
sterilizing the sharp medical device and enclosing in a sterile container; delivering the sterile container to a point of use and removing it from the sterile container;
after use, breaking the frangible connector;
disposing of the sharp medical device in a first waste container for medical sharps;
and disposing of the non-sharp portion in a second waste container.
